(19) United States
(12) Patent Application Publication
(10) Pub. No.: US 2020/0093948 A1
(43) Pub. Date: Mar. 26, 2020

(54) PORTABLE CLEANING DEVICE

(71) Applicant: VirtuOx, Inc., Coral Springs, FL (US)

(72) Inventors: Kyle Miko, Coral Springs, FL (US); Steve Lica, Coral Springs, FL (US); Jon Fedele, Coral Springs, FL (US)

(21) Appl. No.: 16/536,809

(22) Filed: Aug. 9, 2019

Related U.S. Application Data
(60) Provisional application No. 62/734,565, filed on Sep. 21, 2018.

(51) Int. Cl. A61L 2/20; A61L 2/26

(52) U.S. Cl. CPC A61L 2/202; A61L 2/26; A61L 2202/122; A61M 16/06; A61L 2202/15; A61L 2202/16; A61L 2202/24; A61L 2202/14

(57) ABSTRACT

A portable cleaning system includes a cleaning module, a portable zippered bag, and a delivery tube. The portable zippered bag is configured to receive a sleep apnea mask. The delivery tube is coupled at a proximal end to the cleaning module. The delivery tube communicates with the portable zippered bag. The cleaning module produces ozone (O₃), such that O₃ travels from the cleaning module to the portable zippered bag, via the delivery tube, to sterilize the sleep apnea mask.

### PRIORITY CLAIM

This application claims priority to U.S. Provisional Application No. 62/734,565, entitled “Portable Cleaning Device”, filed Sep. 21, 2018, the entire contents of which are incorporated herein by reference and relied upon.

### BACKGROUND

Proper cleaning of medical equipment ensures that the medical equipment operates at the highest level of performance. Furthermore, especially with medical equipment, proper cleaning ensures the highest levels of sterilization and safety. Medical equipment needs to be properly cleaned, whether the medical equipment is utilized in traditional settings or in non-traditional settings, such as for in-home use. Though medical providers, like hospitals and primary care providers, may implement various cleaning protocol for their medical equipment, individuals with in-home use products may be less diligent with respect to properly cleaning their products. One such example of an in-home use product is a sleep apnea machine, such as a continuous positive airway pressure (CPAP) machine or a bi-level positive airway pressure machine.

Specifically with sleep apnea machines, portions of the equipment such as the patient mask, and humidifier water, should typically be cleaned daily. Other portions of the equipment, such as the tubing and humidifier chamber, should typically be cleaned weekly. Though frequent cleaning is important, patients with in-home use products rarely adhere to the required cleaning schedule, which leads to bacteria and mold accumulation within the equipment. Accumulation of mold and bacteria may reduce the performance of the machine and cause various health complications with the patient.

For at least the above reasons, it is desirable to provide improved systems and devices for the cleaning of medical equipment, such as sleep apnea equipment.

### SUMMARY

To improve the medical equipment cleaning paradigm, new cleaning devices, systems, and methods of cleaning are described herein. The present disclosure seeks to implement new devices, systems, and methods for cleaning medical devices, which perform medical device sterilization in an efficient manner. The devices, systems, and methods disclosed herein are self-contained, such that no additional products or adapters are required and no ongoing device maintenance is necessary. Likewise, the devices, systems, and methods disclosed herein are lightweight, portable, and operate with minimal noise, ensuring that sterilization may be performed at any time and in any location. Furthermore, the devices, systems, and methods disclosed herein are easy to use and operate, thus encouraging patients to use the products.

In light of the disclosure herein, and without limiting the scope of the invention in any way, in a first aspect of the present disclosure, which may be combined with any other aspect listed herein unless specified otherwise, a portable cleaning system includes a cleaning module, a portable zippered bag, and a delivery tube. The portable zippered bag is configured to receive a sleep apnea mask. The delivery tube is coupled at a proximal end to the cleaning module. The delivery tube communicates with the portable zippered bag. The cleaning module produces ozone (O₃), such that O₃ travels from the cleaning module to the portable zippered bag, via the delivery tube, to sterilize the sleep apnea mask.

In a second aspect of the present disclosure, which may be combined with any other aspect listed herein unless specified otherwise, a zipper is configured to seal a distal end of the delivery tube in the portable zippered bag.

In a third aspect of the present disclosure, which may be combined with any other aspect listed herein unless specified otherwise, the portable zippered bag further includes a port, such that the distal end of the delivery tube engages with the port.

In a fourth aspect of the present disclosure, which may be combined with any other aspect listed herein unless specified otherwise, the port is threaded, such that the distal end of the delivery tube engages with the port via threaded engagement.

In a fifth aspect of the present disclosure, which may be combined with any other aspect listed herein unless specified otherwise, the cleaning module includes a cleaning timer, such that the cleaning module produces O₃ for an amount of time as dictated by the cleaning timer.

In a sixth aspect of the present disclosure, which may be combined with any other aspect listed herein unless specified otherwise, responsive to the amount of time dictated by the cleaning timer expiring, the cleaning module delivers ambient air from the cleaning module to the portable zippered bag, via the delivery tube.

In a seventh aspect of the present disclosure, which may be combined with any other aspect listed herein unless specified otherwise, the cleaning module includes a maintenance timer, such that the maintenance timer reminds a user to sterilize the sleep apnea mask after an amount of time as dictated by the maintenance timer.

In an eighth aspect of the present disclosure, which may be combined with any other aspect listed herein unless specified otherwise, the portable zippered bag is constructed of a semi-permeable membrane.

In a ninth aspect of the present disclosure, which may be combined with any other aspect listed herein unless specified otherwise, the cleaning module is powered by a rechargeable battery.

In a tenth aspect of the present disclosure, which may be combined with any other aspect listed herein unless specified otherwise, the cleaning module includes a plurality of lights for indicating a cleaning module status to a user.

In an eleventh aspect of the present disclosure, which may be combined with any other aspect listed herein unless specified otherwise, the cleaning module status includes at least one of on/of status, power level status, maintenance status, and sterilization status.

In a twelfth aspect of the present disclosure, which may be combined with any other aspect listed herein unless specified otherwise, a portable cleaning system includes a cleaning module, a sealable cleaning container, and a delivery tube. The delivery tube is coupled at a proximal end to the cleaning module. The delivery tube is disposed, at a distal end, into the sealable cleaning container. Activated oxygen is delivered, from the cleaning module to the sealable cleaning container, via the delivery tube, to clean a device stored in the sealable cleaning container.

In a thirteenth aspect of the present disclosure, which may be combined with any other aspect listed herein unless specified otherwise, the sealable cleaning container is a portable zippered bag.

In a fourteenth aspect of the present disclosure, which may be combined with any other aspect listed herein unless specified otherwise, a zipper is configured to seal the distal end of the delivery tube in the sealable cleaning container.

In a fifteenth aspect of the present disclosure, which may be combined with any other aspect listed herein unless specified otherwise, activated oxygen is ozone (O₃).

In a sixteenth aspect of the present disclosure, which may be combined with any other aspect listed herein unless specified otherwise, the device is a sleep apnea mask.

In a seventeenth aspect of the present disclosure, which may be combined with any other aspect listed herein unless specified otherwise, the delivery tube is a sleep apnea tube.

In an eighteenth aspect of the present disclosure, which may be combined with any other aspect listed herein unless specified otherwise, coupling between the proximal end of the delivery tube and the cleaning module further includes a tube adapter.

In a nineteenth aspect of the present disclosure, which may be combined with any other aspect listed herein unless specified otherwise, responsive to activated oxygen being delivered, ambient air is delivered, from the cleaning module to the sealable cleaning container, via the delivery tube.

In a twentieth aspect of the present disclosure, which may be combined with any other aspect listed herein unless specified otherwise, a method of sleep apnea mask sterilization includes opening a zippered bag and placing a sleep apnea mask into the zippered bag. The method includes coupling a first end of a delivery tube to a cleaning module. The method further includes placing a second end of the delivery tube into the zippered bag and sealing the zippered bag. Finally, the method includes activating the cleaning module, wherein the cleaning module produces ozone (O₃) that travels from the cleaning module to the zippered bag, via the delivery tube, and sterilizing the sleep apnea mask via O₃.

Additional features and advantages of the disclosed devices, systems, and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been principally selected for readability and instructional purposes, and not to limit the scope of the inventive subject matter.

### BRIEF DESCRIPTION OF THE FIGURES

FIG. 1 is a side elevation view of a portable cleaning system, according to an example embodiment of the present disclosure.

FIG. 2 is a side elevation view of a cleaning module, according to an example embodiment of the present disclosure.

FIGS. 3A to 3D are side elevation views of a cleaning module with a display, according to an example embodiment of the present disclosure.

FIGS. 4A to 4F are side elevation views of a method of using a cleaning module with a display, according to an example embodiment of the present disclosure.

### DETAILED DESCRIPTION OF EXAMPLE EMBODIMENTS

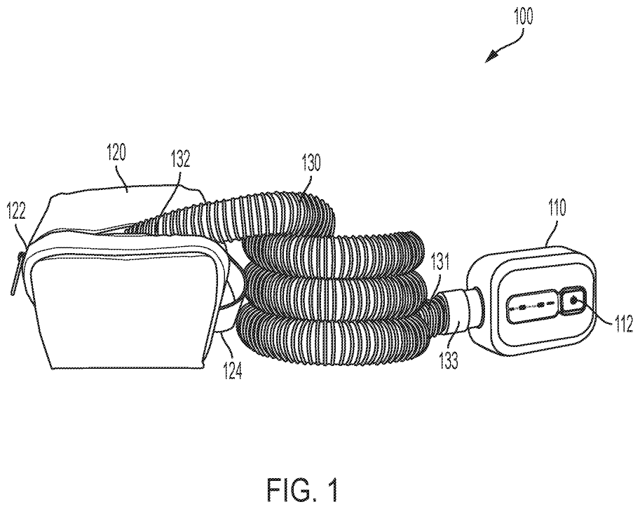

As discussed above, improved devices, systems, and methods for cleaning medical devices are disclosed herein. FIG. 1 illustrates a side elevation view of a portable cleaning system 100, according to an example embodiment of the present disclosure. The portable cleaning system 100 includes a cleaning module 110, a sealed cleaning environment 120, and a delivery tube 130.

In an embodiment, the cleaning module 110 is a portable device. For example, in a preferred embodiment, the cleaning module 110 has a profile of approximately 5 inches by 3 inches. Likewise, for example, in a preferred embodiment, the cleaning module 110 has a weight of approximately 0.5 pounds. Generally, it should be appreciated that sealed cleaning environment 120 is sized and shaped for storing both cleaning module 110 and delivery tube 130 when not being used for sterilization purposes.

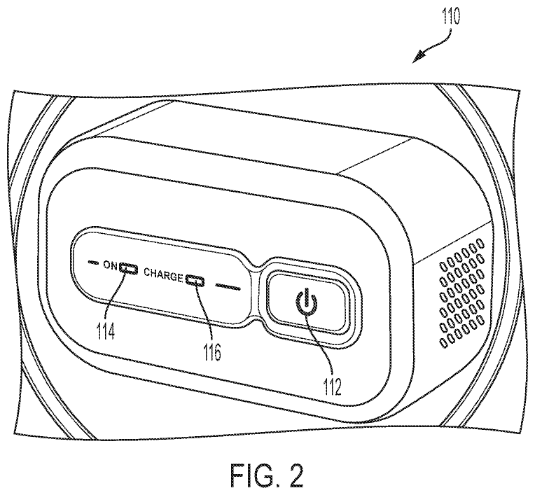

FIG. 2 illustrates a side elevation view of the cleaning module 110, according to an example embodiment of the present disclosure. As illustrated in FIG. 2, the cleaning module 110 includes a power button 112. Cleaning module 110 may further include displays or indicators for various performance metrics. For example, cleaning module 110 may include an on/off indicator 114 and a charge indicator 116. On/off indicator 114 indicates, to the user, whether cleaning module 110 is operating. Charge indicator 116 indicates, to the user, whether cleaning module 110 requires charging and/or new batteries. In an embodiment, charge indicator 116 includes three bars indicating charge level, such as high charge, medium charge, and low charge. In an alternate embodiment, charge indicator 116 implements a color scheme, such as green for high charge, yellow for medium charge, and red for low charge. It should be appreciated that cleaning module 110 may include a number of other indicators, for displaying additional performance metrics, or for other features.

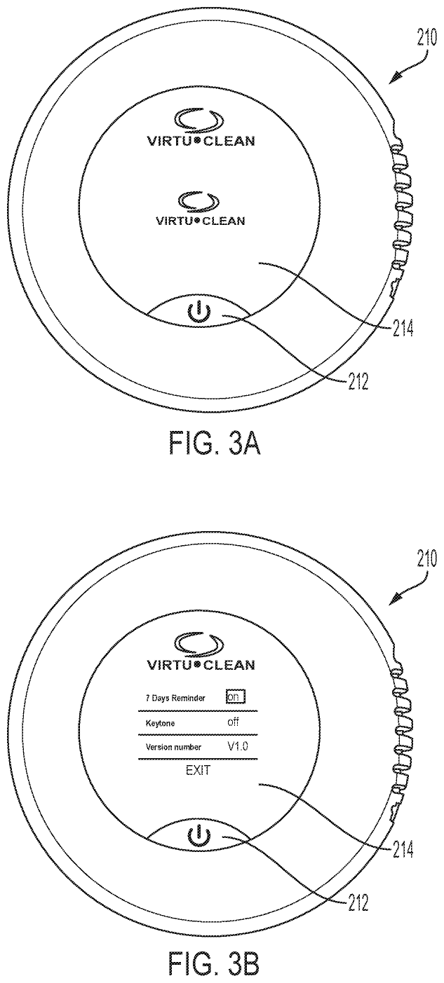

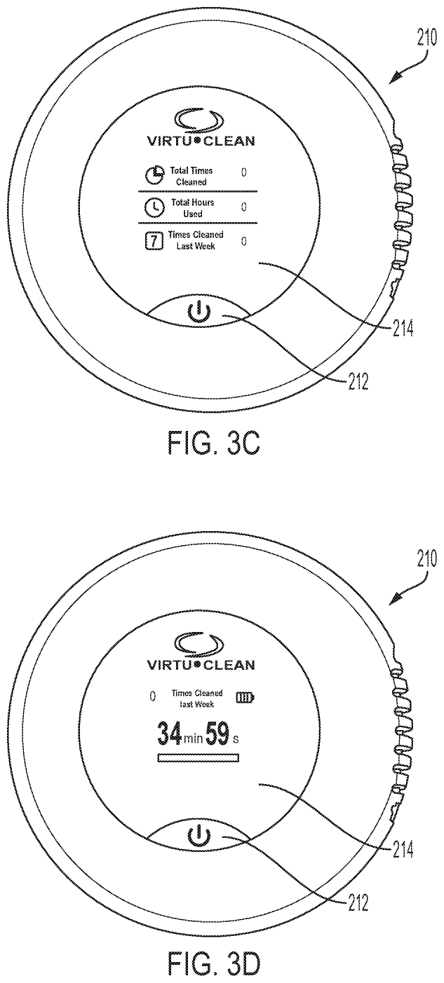

In a different embodiment, as illustrated by FIGS. 3A to 3D, a cleaning module 210 further includes a power button 212 and a LCD display 214. As illustrated by FIG. 3A, the LCD display 214 may indicate, to the user, whether the cleaning module 210 is on or off, and/or whether the cleaning module 210 is charging or charging is complete. LCD display 214 may also provide the user with additional information and customizable features.

For example, FIG. 3B illustrates a custom menu, whereby the user can select a weekly reminder, such that the cleaning module 210 provides an audible alarm every seven days, to alert the user that cleaning is required. The user can select whether audible sounds or keytones are presented. The user is able to cycle through this custom menu by tapping the power button 212. When a particular selection is highlighted, the user can double-tap or hold the power button 212 in order to choose a particular selection.

As illustrated by FIG. 3C, cleaning module 210 may present cleaning statistics to the user via LCD display 214. For example, cleaning module 210 displays the total number of times the user has cleaned products with cleaning module 210, the total number of hours cleaning module 210 has been used, and the total number of times the user has cleaned products within the past seven days.

During a particular cleaning procedure, as illustrated by FIG. 3D, cleaning module 210 may present a countdown timer to the user via LCD display 214. For example, cleaning module 210 displays the time remaining in a particular cleaning cycle, along with the power remaining via a battery indicator. Cleaning module 210 may further indicate the total number of times the user has cleaned products within the past seven days.

In addition to the features described above, LCD display 214 may be used to provide the user with additional information and features. For example, at first use LCD device 214 may display a QR code for the user, which is captured via a smartphone or other internet-connected device. In this way, the user may automatically register the cleaning module 210 for device warranty purposes.

It should be appreciated that either cleaning module 110 or cleaning module 210 can include these features as discussed above. Likewise, more generally, either cleaning module 110 or cleaning module 210 can be implemented into the cleaning systems disclosed in greater detail herein.

Referring back to FIG. 1, in an embodiment, the cleaning module 110 is battery powered. For example, cleaning module 110 may further include a rechargeable lithium ion battery for improved portability. In a different example, cleaning module 110 may include a power cord, configured to engage with an external power source. The power cord may be implemented, with the rechargeable lithium ion battery, for at-home charging. For example, the power cord may couple with the cleaning module 110 via mini-USB, USB, or other similar connections.

The sealed cleaning environment 120 includes a sealing feature 122, such as a zipper, snaps, hook-and-loop fasteners, or other similar fastening component for sealing and unsealing the sealed cleaning environment 120. The sealed cleaning environment 120 further includes a handle 124 for improved portability. In an example embodiment, the sealed cleaning environment 120 is a flexible bag, such as a nylon or polyester bag. In one embodiment, the sealed cleaning environment 120 is non-permeable. In a different embodiment, the sealed cleaning environment 120 is semi-permeable.

The delivery tube 130 has a first end 131 and a second end 132. The delivery tube 130 is configured to be coupled, at the first end 131, to the cleaning module 110. For example, the delivery tube 130 may couple to cleaning module 110 via threaded connection, luer connection, interference fit, or any other related connection for coupling delivery tube 130 to cleaning module 110. In an embodiment, the delivery tube 130 includes a CPAP tube connector for coupling to cleaning module 110. Likewise, the delivery tube 130 is disposed, at the second end 132, into the sealed cleaning environment 120. The sealed cleaning environment 120 is closed, via sealing feature 122, to form a seal around delivery tube 130. In this configuration, the delivery tube 130 forms a contained fluid pathway for air, liquid, or any other fluid, to flow between the cleaning module 110 and the sealed cleaning environment 120.

In an example embodiment, the coupling between the first end 131 of the delivery tube 130 and the cleaning module 110 includes a tube adapter 133, such as a sleep apnea tube adapter. In this example, the tube adapter 133 is further cleaned and sterilized by the cleaning module 110. Likewise, in an example embodiment, the sealed cleaning environment 120 includes a tube adapter (not illustrated), such that the second end 132 of the delivery tube 130 may be coupled directly to the adapter at the sealed cleaning environment 120. For example, the sealed cleaning environment 120 includes a threaded adapter port, configured to engage with the second end 132 of delivery tube 130. In this embodiment, the sealed cleaning environment 120 may still include sealing feature 122, such as a zipper.

To operate system 100, a user places a device that requires cleaning, such as a sleep apnea mask and related components, into the sealed cleaning environment 120. The user attaches the first end 131 of the delivery tube 130 to the cleaning module 110. In an example embodiment, the delivery tube 130 is a sleep apnea tube; in this embodiment, no additional tube is required. The user ensures that the second end 132 of the delivery tube 130 is disposed into the sealed cleaning environment 120 or, alternatively, that the second end 132 of the delivery tube 130 is coupled to a tube adapter. The user will then seal the sealed cleaning environment 120 via sealing feature 122. For example, the user zips up the sealed cleaning environment 120. In this configuration, the sealed cleaning environment 120 is entirely self-contained, except for the portion of the delivery tube 130 that exits the sealed cleaning environment 120 and extends back to the cleaning module 110. The user pushes power button 112 and verifies that the cleaning module 110 is operating, such as via on/off indicator 114.

While cleaning module 110 is operating, it is performing sterilization functions. Namely, cleaning module 110 produces activated oxygen, which in an embodiment includes ozone (O₃). The activated oxygen is delivered, from the cleaning module 110 to the sealed cleaning environment 120, via the delivery tube 130. Once the activated oxygen enters the sealed cleaning environment 120, it performs sterilization on the device, such as the sleep apnea mask and related components stored within the sealed cleaning environment 120. Specifically, the activated oxygen kills germs, bacteria, and mold on any surface that the activated oxygen encounters. In an embodiment, the cleaning module 110 is configured to operate and deliver activated oxygen quietly.

Cleaning module 110 ensures that the pressure of activated oxygen within sealed cleaning environment 120 is greater than ambient pressure outside the sealed cleaning environment 120. In this way, even though the sealed cleaning environment 120 may not be a hermetically sealed environment (e.g., due to air flow across sealing feature 122, such as a zipper), the sealed cleaning environment 120 nonetheless includes a sufficient concentration of activated oxygen for sterilization purposes. In an embodiment, cleaning module 110 further includes a display, such as LCD display 214, for dynamically indicating the percentage of activated oxygen, such as in parts-per-million, which is being delivered from cleaning module 110 to sealed cleaning environment 120. In a related embodiment, the display indicates the percentage of activated oxygen being delivered from cleaning module 110 to sealed cleaning environment 120 in pass/fail terms, such as displaying a green light when the percentage of activated oxygen meets or exceeds a particular level and displaying a red light when the percentage of activated oxygen does not meet the particular level.

After approximately thirty minutes, cleaning module 110 ceases performing sterilization functions. In an embodiment, cleaning module 110 includes a cleaning timer for timing and ceasing sterilization functions. The cleaning timer may communicate with a light (similar to charge indicator 116 previously discussed herein), a speaker, or any other indicators to indicate whether cleaning is or is not taking place. In an embodiment, cleaning timer includes a display for indicating a count down time associated with the time remaining in a particular sterilization cycle.

In an embodiment, once sterilization is finished, the cleaning module 110 automatically shuts off. Alternatively, in an embodiment, once sterilization is finished, the cleaning module 110 may deliver ambient air into the sealed cleaning environment 120, via the delivery tube 130. Delivery of ambient air may dilute any remaining activated oxygen in the sealed cleaning environment 120. Once sterilization functions have finished, the portable cleaning system 100 has killed approximately 99.99% of germs, bacteria, and mold on the device stored within the sealed cleaning environment 120.

In other embodiments, the portable cleaning system 100 may be used to clean other sleep apnea products, such as a humidifier. For example, with a sleep apnea humidifier, the user attaches the first end 131 of the delivery tube 130 to the cleaning module 110 and attaches the second end 132 of the delivery tube 130 directly to the humidifier. The user pushes power button 112 and verifies that the cleaning module 110 is operating, such as via on/off indicator 114. In this embodiment, the activated oxygen is delivered from the cleaning module 110 directly into the humidifier, via the delivery tube 130.

As implemented and described above, the portable cleaning system 100 does not require any filters or other adapters. Likewise, the portable cleaning system 100 does not require any washing of the components (e.g., sleep apnea mask, delivery tube 130, and related components) with soap, water, or other cleaning solutions.

As previously noted, cleaning module 110 may include additional indicators for other features. In an embodiment, cleaning module 110 includes a maintenance timer, similar to the cleaning timer previously described herein. The maintenance timer may communicate with a light (similar to charge indicator 116 previously discussed herein), a speaker, or any other indicators to indicate whether maintenance is or is not required. Maintenance timer may be configured to remind the user to sterilize components, such as sleep apnea mask and delivery tube 130, at regular intervals. For example, maintenance timer may alert the user every seven days for weekly cleaning of components. In an embodiment, maintenance timer alerts the user every day for daily cleaning of the sleep apnea mask, such as via a first light on cleaning module 110; maintenance timer, likewise, alerts the user every week for weekly cleaning of the delivery tube 130, such as via a second light on cleaning module 110. In an embodiment, maintenance timer further includes a display that indicates the last time that cleaning module 110 was used.

In an embodiment described above, the portable cleaning system 100 is applicable for in-home use, such as for sterilizing one sleep apnea mask, delivery tube 130, and related components. In an alternate embodiment, the portable cleaning system 100 may be implemented in a hospital setting, such as for sterilizing multiple sleep apnea masks, delivery tubes, and related components. It should be appreciated that, when implemented in a hospital setting, sealed cleaning environment 120 may be larger, so as to accommodate more devices.

Furthermore, while the examples herein relate to sterilization of sleep apnea components, it should be appreciated that system 100 may be used with any components that require sterilization.

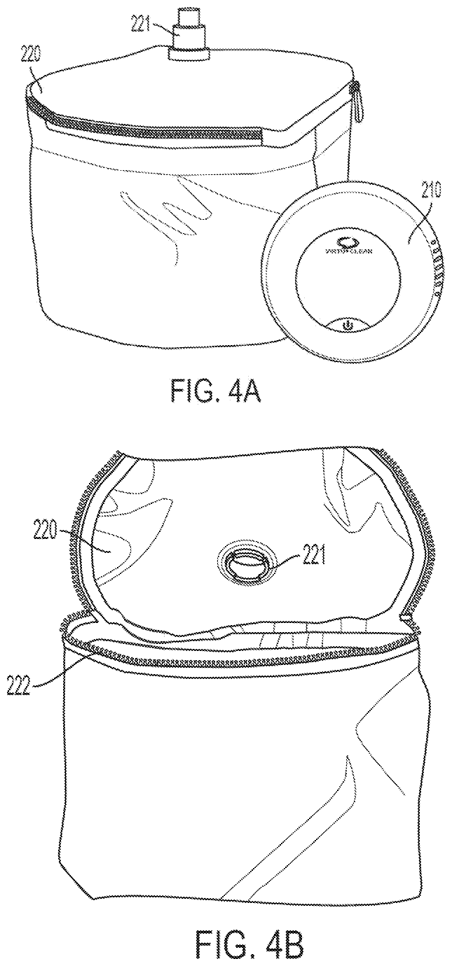

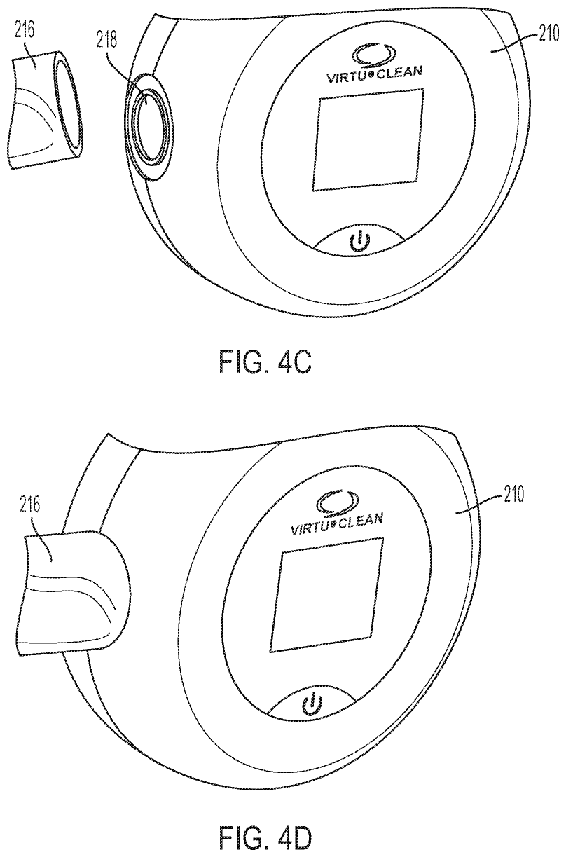

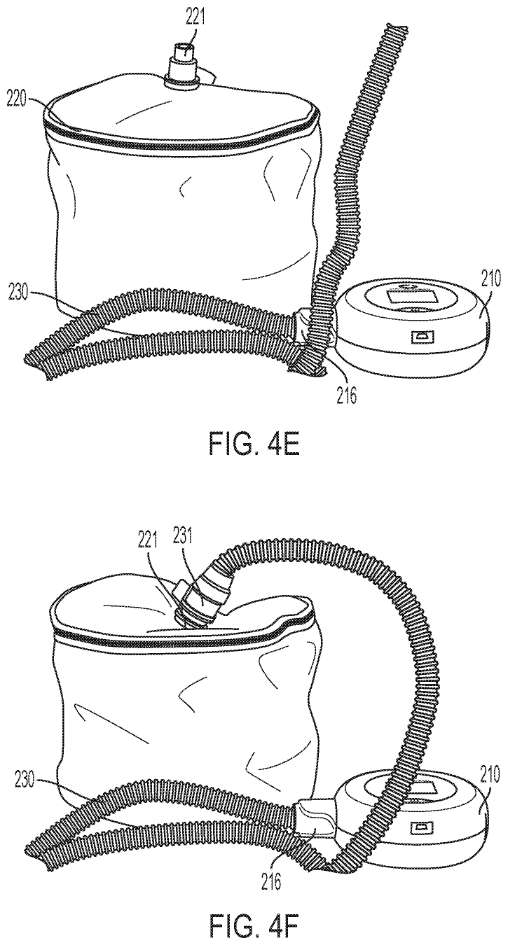

FIGS. 4A to 4F illustrate a typical method of using for a cleaning module, such as cleaning module 110 or cleaning module 210 with LCD display 214. For example, with reference to FIGS. 4A and 4B, in an embodiment, cleaning module 210 is provided, along with sealed cleaning environment 220. Sealed cleaning environment 220 includes a tube adapter 221 and a sealing feature 222. Tube adapter 221 may provide connection to additional components, such as a sleep apnea tube. Sealing feature 222 may be a zipper fastener, button fastener, hook-and-loop fastener, or any other related components for sealing the sealed cleaning environment 220.

Sleep apnea components, or other components for sterilization, are placed within the sealed cleaning environment 220. The user then seals this sealed cleaning environment 220, via sealing features 222.

With reference to FIGS. 4C and 4D, the user connects cleaning module 210 to a first end 216 of a tube, such as a sleep apnea tube. Specifically, cleaning module 210 includes a recessed connection 218, such that the first end 216 of the tube engages with the recessed connection 218. In an example, each of the recessed connection 218 and the first end 216 of the tube are threaded. It should be appreciated, however, that other connection features are contemplated.

With reference to FIGS. 4E and 4F, the user connects the tube 230 to the sealed cleaning environment 220. More particularly, a second end 231 of the tube 230 engages with tube adapter 221. In an example, each of tube adapter 221 and the second end 231 of the tube 230 are threaded. It should be appreciated, however, that other connection features are contemplated.

Once the tube 230 is connected to each of the sealed cleaning environment 220 and the cleaning module 210, the sleep apnea components inside sealed cleaning environment 220 may be cleaned. Specifically, for example, the cleaning module 220 is activated, such that it produces ozone (O₃). The ozone travels from the cleaning module 210 to the sealed cleaning environment 220, via the tube 230.

The many features and advantages of the present disclosure are apparent from the written description, and thus, the appended claims are intended to cover all such features and advantages of the disclosure. Further, since numerous modifications and changes will readily occur to those skilled in the art, the present disclosure is not limited to the exact construction and operation as illustrated and described. Therefore, the described embodiments should be taken as illustrative and not restrictive, and the disclosure should not be limited to the details given herein but should be defined by the following claims and their full scope of equivalents, whether foreseeable or unforeseeable now or in the future.

1. A portable cleaning system comprising:
a cleaning module;
a portable zippered bag, configured to receive a sleep apnea mask;
a delivery tube, wherein the delivery tube is coupled at a proximal end to the cleaning module, and wherein the delivery tube communicates with the portable zippered bag,
wherein the cleaning module produces ozone (O₃), such that O₃ travels from the cleaning module to the portable zippered bag, via the delivery tube, to sterilize the sleep apnea mask.

2. The portable cleaning system of claim 1, wherein a zipper is configured to seal a distal end of the delivery tube in the portable zippered bag.

3. The portable cleaning system of claim 1, wherein the portable zippered bag further includes a port, such that the distal end of the delivery tube engages with the port.

4. The portable cleaning system of claim 3, wherein the port is threaded, such that the distal end of the delivery tube engages with the port via threaded engagement.

5. The portable cleaning system of claim 1, wherein the cleaning module includes a cleaning timer, such that the cleaning module produces O₃ for an amount of time, as dictated by the cleaning timer.

6. The portable cleaning system of claim 6, wherein, responsive to the amount of time dictated by the cleaning timer expiring, the cleaning module delivers ambient air from the cleaning module to the portable zippered bag, via the delivery tube.

7. The portable cleaning system of claim 1, wherein the cleaning module includes a maintenance timer, such that the maintenance timer reminds a user to sterilize the sleep apnea mask after an amount of time as, dictated by the maintenance timer.

8. The portable cleaning system of claim 1, wherein the portable zippered bag is constructed of a semi-permeable membrane.

9. The portable cleaning system of claim 1, wherein the cleaning module is powered by a rechargeable battery.

10. The portable cleaning system of claim 1, wherein the cleaning module includes a plurality of lights for indicating a cleaning module status to a user.

11. The portable cleaning system of claim 10, wherein the cleaning module status includes at least one of on/of status, power level status, maintenance status, and sterilization status.

12. A portable cleaning system comprising:
a cleaning module;
a sealable cleaning container; and
a delivery tube, wherein the delivery tube is coupled at a proximal end to the cleaning module, and wherein the delivery tube is disposed, at a distal end, into the sealable cleaning container,
wherein activated oxygen is delivered, from the cleaning module to the sealable cleaning container, via the delivery tube, to clean a device stored in the sealable cleaning container.

13. The portable cleaning system of claim 12, wherein the sealable cleaning container is a portable zippered bag.

14. The portable cleaning system of claim 13, wherein a zipper is configured to seal the distal end of the delivery tube in the sealable cleaning container.

15. The portable cleaning system of claim 12, wherein activated oxygen is ozone (O₃).

16. The portable cleaning system of claim 12, wherein the device is a sleep apnea mask.

17. The portable cleaning system of claim 12, wherein the delivery tube is a sleep apnea tube.

18. The portable cleaning system of claim 12, wherein coupling between the proximal end of the delivery tube and the cleaning module further includes a tube adapter.

19. The portable cleaning system of claim 12, wherein, responsive to activated oxygen being delivered, ambient air is delivered, from the cleaning module to the sealable cleaning container, via the delivery tube.

20. A method of sleep apnea mask sterilization comprising:
opening a zippered bag;
placing a sleep apnea mask into the zippered bag;
coupling a first end of a delivery tube to a cleaning module;
placing a second end of the delivery tube into the zippered bag;
sealing the zippered bag;
activating the cleaning module, wherein the cleaning module produces ozone (O₃) that travels from the cleaning module to the zippered bag, via the delivery tube; and
sterilizing the sleep apnea mask via O₃.
